(19) United States
(12) Patent Application Publication
(10) Pub. No.: US 2021/0282812 A1
(43) Pub. Date: Sep. 16, 2021

(54) PULL CHORD MANUALLY DRIVEN INTRAOSSEOUS INJECTION DEVICES AND METHODS

(71) Applicant: TELEFLEX LIFE SCIENCES LIMITED, Valletta (MT)

(72) Inventors: Morgan TIERNEY, Ferbane (IE); Alex FOMINAS, Athlone (IE); Eugene SKELTON, Dublin (IE); Anthony WRIGHT, Dungarvan (IE)

(21) Appl. No.: 17/331,385

(22) Filed: May 26, 2021

Related U.S. Application Data
(63) Continuation of application No. PCT/IB2019/060250, filed on Nov. 27, 2019.
(60) Provisional application No. 62/772,241, filed on Nov. 28, 2018.

(51) Int. Cl. A61B 17/34

(52) U.S. Cl. CPC A61B 17/3472; A61B 2017/00407

(57) ABSTRACT

Manually driven intraosseous devices and associated methods are disclosed. The intraosseous device includes a housing and a transmission at least partially disposed within the housing. The transmission includes a first portion and a second portion. The second portion including a manual actuator that is at least partially disposed outside of the housing. The intraosseous device also includes a penetrator assembly releasably connected to the first portion of the transmission. The transmission is configured to transmit a force, which is applied manually to the manual actuator, to the penetrator assembly to rotate the penetrator assembly relative to the housing and to drive the penetrator assembly through a bone into an intraosseous space. The manual actuator is a pull chord and the force is configured to be applied by an operator to a first end of the pull chord to unwind the pull chord and transmit the force to the penetrator assembly.

### CROSS-REFERENCE TO RELATED APPLICATIONS

This application is a continuation of International Patent Application PCT/IB2019/060250, filed Nov. 27, 2019, which claims the benefit of priority to U.S. Provisional Patent Application No. 62/772,241, filed Nov. 28, 2018, the disclosures of which are incorporated herein by reference in their entirety.

### TECHNICAL FIELD

This application relates generally to systems, devices, and methods for intraocular injection.

### BACKGROUND

Every year, patients around the world are treated for life-threatening emergencies. Such life-threatening emergencies include, for example, shock, trauma, cardiac arrest, drug overdoses, diabetic ketoacidosis, arrhythmias, burns, and status epilepticus. According to one study, more than 1.5 million patients suffer from heart attacks (myocardial infarctions) each year, with over 500 thousand of those patients dying from complications arising from the heart attacks. Military, remote, and/or rural environments may present unique challenges for treatment of life-threatening injuries. Many patients die unnecessarily because intravenous (IV) access cannot be achieved in a timely manner.

An essential element for treating many of the above-described life threatening emergencies is rapid establishment of an IV line in order to administer drugs and fluids directly into a patient's vascular system. Whether in an ambulance by paramedics, in an emergency room by emergency specialists, in a remote or rural area by a first-responder, or on a battlefield by a medic, the goal is the same—quickly start an IV in order to administer lifesaving drugs and fluids. To a large degree, the ability to successfully treat most critical emergencies is dependent on the skill and/or luck of an operator to rapidly access the vascular system. While it is relatively easy to start an IV on some patients, medical professionals often experience significant difficulty establishing IV access for a number of patients, for whom the success rate of IV establishment is particularly low. In addition to the risk to life and limb, failed attempts to establish an IV can lead to unnecessary pain to patients being probed.

In the case of patients with chronic disease or the elderly, availability of easily accessible veins may be depleted. Other patients may have no available IV sites due to anatomical scarcity of peripheral veins, obesity, extreme dehydration or previous IV drug use. For such patients, finding a suitable site for administering lifesaving therapy often becomes a monumental and frustrating task. While morbidity and mortality statistics are not generally available, it is generally known that many patients with life threatening emergencies have died because access to the vascular system with lifesaving IV therapy was delayed or simply not possible.

The intraosseous (IO) space provides a direct conduit to the vascular system and provides an attractive alternate route for the administration of IV drugs and fluids. Drugs and/or fluid administered into an IO space enter the blood circulation system at least as rapidly as when administered intravenously. Accordingly, bone marrow may be an analog of a large, non-collapsible vein. IO infusion has long been the standard of care in pediatric emergencies when rapid IV access is not possible.

### SUMMARY

The present inventors recognize that there is a need to improve one or more features of IO injection devices and methods. For example, there exists a need for IO injection devices that are manually driven and have a low cost of production for use as an alternative to IV access in emergent, urgent, or medically necessary cases.

An aspect of the various embodiments of the invention is directed to an intraosseous device. The intraosseous device includes a housing and a transmission at least partially disposed within the housing. the transmission includes a first portion and a second portion. The second portion of the transmission may include a manual actuator that is at least partially disposed outside of the housing. The intraosseous device further includes a penetrator assembly releasably connected to the first portion of the transmission. The transmission is configured to transmit a force, which is applied manually to the manual actuator, to the penetrator assembly to rotate the penetrator assembly relative to the housing and to drive the penetrator assembly through a bone into an intraosseous space.

Another aspect of the present invention includes a method of accessing an intraosseous space of a patient. The method includes providing an intraosseous device comprising a housing and a transmission at least partially disposed within the housing. The transmission includes a first portion and a second portion. The provided intraosseous device further includes a penetrator assembly releasably connected to the first portion of the transmission. The method also includes driving the penetrator assembly through a bone into the intraosseous space of the patient. Driving the penetrator assembly through the bone into the intraosseous space of the patient includes manually applying a first force to the housing, where the first force directs the intraosseous device towards an insertion site on the patient. Driving the penetrator assembly through the bone into the intraosseous space of the patient further includes manually applying a second force, concurrently with manually applying the first force, to the second portion of the transmission and transmitting the second force to the penetrator assembly to rotate the penetrator assembly relative to the housing.

There are, of course, additional aspects of the various embodiments of the invention disclosed herein that will be described below and which will form the subject matter of the claims. In this respect, before explaining at least one aspect of the invention in detail, it is to be understood that the invention is not limited in its application to the details of construction and to the arrangements of the components set forth in the following description or illustrated in the drawings. The invention is capable of aspects in addition to those described and of being practiced and carried out in various ways. Also, it is to be understood that the phraseology and terminology employed herein, as well as the Abstract, are for the purpose of description and should not be regarded as limiting.

As such, those skilled in the art will appreciate that the conception upon which this invention is based may readily be utilized as a basis for the designing of other structures, methods, and systems for carrying out the several purposes of the invention. It is important, therefore, that the claims be regarded as including such equivalent constructions insofar as they do not depart from the spirit and scope of the invention.

### BRIEF DESCRIPTION OF DRAWINGS

In order that the invention may be readily understood, aspects of the invention are illustrated by way of examples in the accompanying drawings; however, the subject matter is not limited to the disclosed aspects.

FIGS. 1A-1D illustrate embodiments of the intraosseous device including a plurality of exemplary housings in accordance with aspects of the invention.

FIGS. 2A and 2B respectively illustrate a prospective view and a cross-sectional prospective view of an exemplary embodiment of the intraosseous device including a reusable transmission in accordance with aspects of the invention.

FIG. 3 illustrates an exploded view of the exemplary embodiment of the intraosseous device shown in FIGS. 2A and 2B in accordance with aspects of the invention.

FIGS. 4A and 4B respectively illustrate a prospective view and a cross-sectional side view of another exemplary embodiment of the intraosseous device including a single-use transmission in accordance with aspects of the invention.

FIG. 5 illustrates an exploded view of the exemplary embodiment of the intraosseous device shown in FIGS. 4A and 4B in accordance with aspects of the invention.

FIGS. 6A-6C illustrate views of an exemplary penetrator assembly of intraosseous devices in accordance with aspects of the invention.

FIG. 7 illustrates an exemplary process for accessing an intraosseous space of a patient in accordance with aspects of the invention.

Features of the intraosseous devices and associated methods according to aspects of the invention are described with reference to the drawings, in which like reference numerals refer to like parts throughout.

### DETAILED DESCRIPTION OF ILLUSTRATIVE EMBODIMENTS OF THE INVENTION

Vascular system access may be essential for treatment of many serious diseases, chronic conditions and acute emergency situations. Yet, many patients experience extreme difficulty obtaining effective treatment because of inability to obtain or maintain intravenous (IV) access. An intraosseous (IO) space provides a direct conduit to a patent's vascular system and systemic circulation. Therefore, IO access is an effective route to administer a wide variety of drugs, other medications and fluids. Rapid IO access offers great promise for almost any serious emergency that requires vascular access to administer lifesaving drugs, other medications and/or fluids when traditional IV access is difficult or impossible.

An intraosseous space may generally be described as region where cancellous bone and associated medullary cavity combine. Bone marrow typically includes blood, blood forming cells, and connective tissue found in an intraosseous space surrounded by compact bone.

IO access may be used as a “bridge” (temporary fluid and drug therapy) during emergency conditions until conventional IV sites can be found and used. Conventional IV sites often become available because fluids and/or medication provided via IO access may stabilize a patient and expand veins and other portions of a patient's vascular system. IO devices and associated procedures incorporating teachings of the present disclosure may become standard care for administering medications and fluids in situations when IV access is difficult or not possible.

Intraosseous access may be used as a “routine” procedure with chronic conditions that substantially reduce or eliminate availability of conventional IV sites. Examples of such chronic conditions may include, but are not limited to, dialysis patients, patients in intensive care units and epilepsy patients. Intraosseous devices and associated apparatus incorporating teachings of the present disclosure may be quickly and safely used to provide IO access to a patient's vascular system in difficult cases such as status epilepticus to give medical personnel an opportunity to administer crucial medications and/or fluids.

The term “driver” may be used in this application to include any type of manual driver satisfactory for inserting a penetrator assembly or an IO needle into selected portions of a patient's vascular system. Various techniques may be satisfactorily used to releasably engage or a penetrator assembly with manual drivers.

For some applications a manual driver may be securely attached to a portion of an IO device or may be formed as an integral component of an IO device. Various types of connectors may also be used to releasably couple a manual driver with a penetrator assembly. A wide variety of connectors and associated connector receptacles, fittings and/or other types of connections with various dimensions and configurations may be satisfactorily used to engage a penetrator assembly with a manual driver.

For some applications a layer or coating (not expressly shown) of an anticoagulant such as, but not limited to, heparin may be placed on interior and/or exterior portions of a catheter or cannula to prevent thrombotic occlusion of the catheter or cannula. Anticoagulants may reduce platelet adhesion to interior surfaces of the catheter or cannula and may reduce clotting time of blood flowing into and through the catheter or cannula. Placing a layer of an anticoagulant on the exterior of a catheter or cannula adjacent to an associated tip may be helpful to prevent clotting.

The term “fluid” may be used in this application to describe any liquid including, but not limited to, blood, water, saline solutions, IV solutions, plasma or any mixture of liquids, particulate matter, dissolved medication and/or drugs appropriate for injection into bone marrow or other insertion sites. The term “fluid” may also be used within this patent application to include body fluids such as, but not limited to, blood and cells which may be withdrawn from an insertion site.

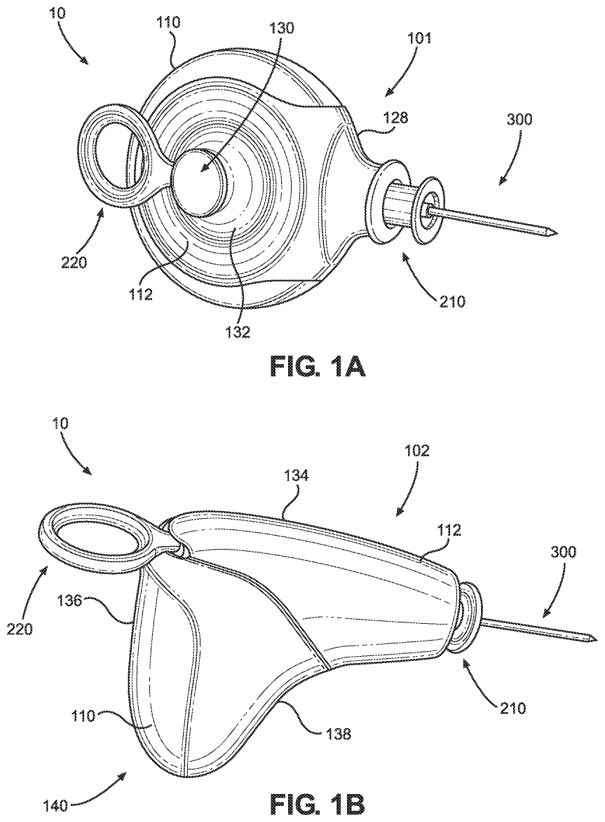

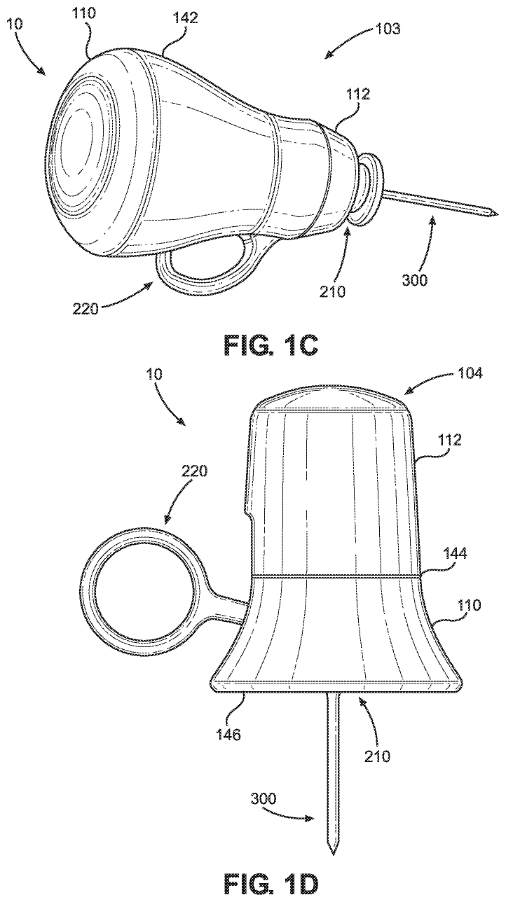

As shown, for example, in FIGS. 1A-1D, an intraosseous (IO) device 10 in accordance with aspects of the invention may include a driver comprising a housing and a transmission at least partially disposed within the housing. The transmission may include a first portion 210 and a second portion 220. The second portion 220 may include a manual actuator (e.g., a pull chord 222) that is at least partially disposed outside of the housing. The IO device 10 may further include a penetrator assembly 300 that may be releasably connected to the first portion 210 of the transmission. The transmission may transmit a force, which is applied manually to the manual actuator, to the penetrator assembly 300 to rotate the penetrator assembly 300 relative to the housing and to drive the penetrator assembly 300 through bone (e.g., the proximal humerus, proximal tibia, distal tibia, etc.) into an intraosseous space (not shown). That is, the transmission may rotate the penetrator assembly 300 relative to the housing to facilitate driving the penetrator assembly 300 through bone into the intraosseous space while an operator presses the IO device 10 through an insertion site. According to aspects of the invention, the manually driven transmission may obviate the need for a powered transmission, which may lower production costs of the IO device 10 and thereby improve patient access to life-saving IO devices 10. In addition, the housing of the IO device 10 may be shaped to improve an operator's view of an insertion site on a patient. Further, the IO device 10 may be rotated about a longitudinal direction of the penetrator assembly 300 in accordance with operator preference such that an operator may manually apply the force to the second portion 220 of the transmission using either the operator's left or right hand.

The housing may be formed of any suitable material, or combination of materials, such as, e.g., plastics, metals, composites, rubber, etc. The housing may include ergonomic portions 110 that may improve usability of the IO device 10 by, e.g., by improving an operator's grip on the IO device 10. The ergonomic portions 110 may be textured. The ergonomic portions 110 may additionally or alternatively be formed of a material different from materials forming other portions 112 of the housing. For example, the housing may include other portions 112 (e.g., structural regions) that are distinct from the ergonomic portion 110. The other portions 112 may be made of a first material and the ergonomic portions 110 may be made of a second material. The first material may be different from the second material. For example, the first material may be a rubber material and the second material may be plastic, metal, and/or composite material.

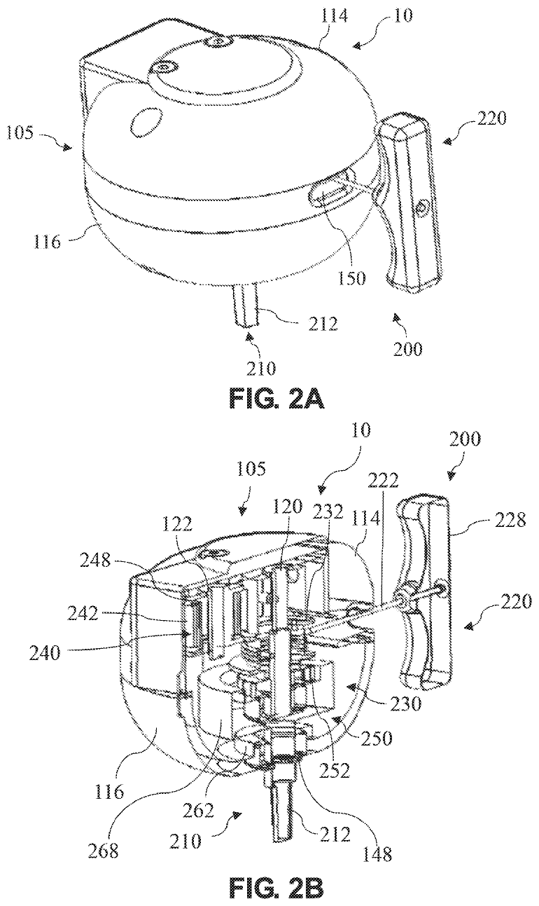

As shown, for example, in FIGS. 2-5, the housing may be divided into one or more bodies. For example, the housing may include a first body 114 and a second body 116. The first body 114 and the second body 116 may be releasably connected together. For example, the first body 114 and the second body 116 may be releasably connected via nuts 124 and bolts 126. The first body 114 and the second body 116 may additionally or alternatively be connected via a snap fit, threads, fixtures, screws, etc. The first body 114 and the second body 116 may alternatively be fixedly connected together. For example, the first body 114 and the second body 116 may be fixed together via an adhesive, weld, etc. The housing may further include a bracket 118 to connect aspects of the transmission, as described below. The housing may also include a first housing pin 120 and a second housing pin 122 that may be mounted to the bracket 118. The bracket 118 may be fixed to the first body 114 by, e.g., nuts 124 and bolts 126.

The housing of the IO device 10 may be uniquely shaped to suit operator preferences and/or for particular applications. For example, according to an exemplary embodiment of the invention depicted in FIG. 1A, the IO device 10 may include a circularly shaped housing 101. More particularly, the circularly shaped housing 101 may be a disc. The circularly shaped housing 101 may include an outer circumferential surface 128, a central region 130, and a side face 132. The first portion 210 of the transmission and may project from the outer circumferential surface 128 of the circularly shaped housing 101. The second portion 220 of the transmission may project from the side face 132 at the central region 130. The ergonomic portions 110 may be arranged at the outer circumferential surface 128.

As shown in FIG. 1B, the IO device 10 may include a triangularly shaped housing 102. The triangularly shaped housing 102 may include a first side 134, a second side 136, and a third side 138. The first portion 210 of the transmission may project from the triangularly shaped housing 102 between the first side 134 and the third side 138. The second portion 220 of the transmission may project from the triangularly shaped housing 102 between the first side 134 and the second side 136. The first side 134 may be arranged substantially along the longitudinal direction of the penetrator assembly 300 such that when the IO device 10 is in use the first side 134 does not obscure a view of a penetrator assembly insertion site. The second side 136 and the third side 138 may together form a grip 140 that may be shaped to fit in an operator's palm. The ergonomic portions 110 may be arranged at the grip 140.

As shown in FIG. 1C, the IO device 10 may include a knob-shaped housing 103. The first portion 210 and the second portion 220 of the transmission may project from the knob-shaped housing 103 at an obtuse angle relative to each other. Accordingly, the force may be applied to second portion 220 of the transmission and the second portion 220 of the transmission may be moved in a direction towards the operator and away from the penetrator assembly 300 to improve the usability of the IO device 10. The ergonomic portions 110 may be arranged around an outer surface 142 of the knob-shaped housing 103.

As shown in FIG. 1D, the IO device 10 may include a bell-shaped housing 104. The first portion 210 and the second portion 220 of the transmission may project from the bell-shaped housing 104 at an obtuse angle relative to each other. Accordingly, the force may be applied to the second portion 220 of the transmission and the second portion 220 of the transmission may be moved in a direction towards the operator and away from the penetrator assembly 300 to improve the usability of the IO device 10. The ergonomic portions 110 may be arranged around an outer surface 144 of the bell-shaped housing 104. The bell-shaped housing 104 may further include a platform 146, which may be placed on the skin of a patient to stabilize the IO device 10 during use.

As shown in FIGS. 2-5, the IO device 10 may include a ball-shaped housing 105. The ball-shaped housing 105 may include a first opening 148 and a second opening 150. The first portion 210 of the transmission may project from the first opening 148. The second portion 220 of the transmission may project from the second opening 150. The first opening 148 and the second opening 150 may be offset from each other by 90 degrees such that the first portion 210 and the second portion 220 of the transmission project from the ball-shaped housing 105 perpendicularly relative to each other. The first opening 148 and the second opening 150 may alternatively be offset from each other by an angle greater than 90 degrees. FIGS. 2-5 show details of embodiments of the transmission of the IO device 10 combined with the ball-shaped housing 105 embodiment. Nevertheless, because embodiments of the transmission of the IO device 10 may be combined with any of the above-described housing embodiments, the ball-shaped housing 105 may simply be referred to as “the housing” when describing structural relationships between the housing and the transmission to emphasize that the transmission may be used with any of the housing embodiments.

The transmission may be any system comprising, e.g., shafts, gears, torque converters, etc. that may transmit manually applied force to the penetrator assembly 300 to rotate the penetrator assembly 300 relative to the housing and to drive the penetrator assembly 300 through the bone and into the intraosseous space. For example, the first portion 210 of the transmission may include a shaft 212. The shaft 212 may interface with the penetrator assembly 300 to connect the transmission to the penetrator assembly 300 to transmit the manual force to the penetrator assembly 300. The shaft 212 may be keyed to connect to the penetrator assembly 300 in a manner that allows the shaft 212 to transmit rotation force to the penetrator assembly 300. For example, the shaft 212 may be five-sided. In addition, the shaft 212 may be magnetic to facilitate connection with the penetrator assembly 300. For example, the shaft 212 may be formed of a magnetic material such as metal.

The manual actuator may be a pull chord 222. Initially, the pull chord 222 may be wound. The manual force may be linearly applied by a hand (i.e., a right or a left hand) of an operator to a first end 224 of the pull chord 222 to unwind the pull chord 222 and transmit the linear manual force to the penetrator assembly 300. Unwinding the pull chord 222 may convert the manual force from a linear force to a rotational force. The pull chord 222 may be formed of any material suitable to transmit the manual force to the penetrator assembly 300 while having sufficient pliability such that the pull chord 222 may be wound, as described herein.

The second portion 220 of the transmission may further include a handle 228 connected to the first end 224 of the pull chord 222. The handle 228 may include be of any shape, size, or material that is suitable to be gripped by a hand of an operator. For example, the handle 228 may be bar-shaped and the first end 224 of the pull chord 222 may be connected to a center of the handle 228. The handle 228 may further include a plurality of recesses 229 arranged along a length of the handle 228 for receiving an operator's fingers. The handle 228 may alternatively be ring-shaped and the ring may be sized to accommodate an operator's finger(s) therein.

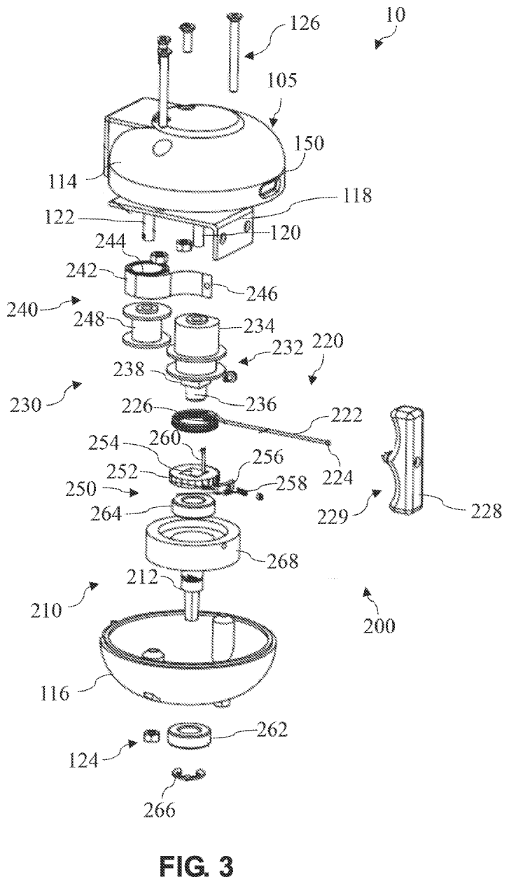

As is particularly shown in FIGS. 2A, 2B, and 3, the IO device 10 may be equipped with a reusable transmission 200. Though the reusable transmission 200 is shown with the IO device 10 including the ball-shaped housing 105, the reusable transmission 200 can be used with any of the disclosed housing embodiments. The reusable transmission 200 may include a third portion 230 that is configured to automatically retract and/or rewind the pull chord 222 and thereby facilitate reuse of the IO device 10. The third portion 230 may be disposed within the housing. The third portion 230 may include a pull chord spool 232 around which the pull chord 222 may be wound. A second end 226 of the pull chord 222 may be fixed to the pull chord spool 232. The pull chord spool 232 may include a first end 234 and a second end 236. The first end 234 of the pull chord spool 232 may be connected to the housing in a manner that allows the pull chord spool 232 to rotate relative to the housing. For example, the first housing pin 120 may connect the first end 234 of the pull chord spool 232 to the housing to facilitate rotation between the pull chord spool 232 and the housing.

The third portion 230 may also include a spring assembly 240 that may bias the pull chord spool 232 in a wound position at which the pull chord 222 is wound a predetermined amount around the pull chord spool 232. The spring assembly 240 may include, e.g., a spring 242 and a spring spool 248. The spring spool 248 may be connected to the housing in a manner that allows the spring spool 248 to rotate relative to the housing. For example, the second housing pin 122 may connect the spring spool 248 to the housing to facilitate rotation between the spring spool 248 and the housing. A first end 244 of the spring 242 may be fixed to the spring spool 248. In the wound position and starting from the first end 244, the spring 242 may be wound a predetermined amount around the spring spool 248. A second end 246 of the spring 242 may be fixed to the pull chord spool 232. The second end 246 of the spring 242 may be fixed to the pull chord spool 232 such that when the pull chord spool 232 rotates due to an unwinding of the pull chord 222 from the pull chord spool 232, starting from the second end 246, the spring 242 may be progressively wound around the pull chord spool 232 and unwound from the spring spool 248. The spring 242 may be biased in the wound position such that when the manual force is released from the pull chord 222, the spring 242 may automatically rewind around the spring spool 248 and may in turn cause the pull chord spool 232 to rotate and rewind the pull chord 222 around the pull chord spool 232. Accordingly, the spring assembly 240 may bias the pull chord spool 232 in the wound position and may automatically rewind the pull chord 222 around the pull chord spool 232.

The third portion 230 may further include a ratchet 250. The ratchet 250 may include, e.g., a sprocket wheel 252, a pawl 256, a pawl spring 258, a ratchet pin 260, a first bearing 262, a second bearing 264, a clip 266, and/or a pawl housing 268. The ratchet 250 may connect the pull chord spool 232 to the shaft 212 such that pull chord spool 232 rotates together with and transfers the manual force to the shaft 212 when the pull chord 222 is unwound and such that the pull chord spool 232 may rotate independently from and relative to the shaft 212 when the pull chord 222 is rewound.

The sprocket wheel 252 may be connected to the pull chord spool 232 such that the sprocket wheel 252 and the pull chord spool 232 rotate together in both clockwise and counterclockwise directions. For example, the pull chord spool 232 may include a keyed region 238 (e.g., square-shaped shaft portion) that may fit within a complementary keyed region 254 of the sprocket wheel 252 (e.g., square-shaped opening) to connect the pull chord spool 232 to the sprocket wheel 252. The keyed region 238 of the pull chord spool 232 may be disposed between the first end 234 and the second end 236 of the pull chord spool 232.

The first bearing 262, the sprocket wheel 252, the pawl 256, the pawl spring 258, and/or the ratchet pin 260 may be disposed at least partially within the pawl housing 268. The second end 236 of the pull chord spool 232 and/or the sprocket wheel 252 may be connected to the first bearing 262 to reduce friction and to facilitate rotation of the pull chord spool 232 and the sprocket wheel 252 relative to the housing and/or the pawl housing 268. The ratchet pin 260 may pivotally mount the pawl 256 to the pawl housing 268. The pawl spring 258 may bias the pawl 256 towards the sprocket wheel 252. The pawl housing 268 may be connected to the shaft 212 such that the pawl housing 268 and the shaft 212 rotate together in both clockwise and counterclockwise directions. The shaft 212 and the pawl housing 268 may be integral or may be distinct structures connected together in a known manner. The pawl housing 268 and/or the shaft 212 may be connected to the second bearing 264, and the second bearing 264 may be connected to the housing. The second bearing 264 may reduce friction and facilitate rotation of the pawl housing 268 and/or the shaft 212 relative to the housing. The clip 266 may be clipped to the pawl housing 268 and/or the shaft 212 to hold components of the reusable transmission 200 at predetermined positions within the housing.

The pawl 256 may be shaped and arranged within the pawl housing 268 such that when the sprocket wheel 252 rotates in a first direction (e.g., the clockwise direction) the pawl 256 may be pushed by the pawl spring 258 into meshed engagement with the sprocket wheel 252. As a result of meshed engagement between the sprocket wheel 252 and the pawl 256, the pawl housing 268 and the shaft 212 may rotate together with the sprocket wheel 252 and the pull chord spool 232 when the sprocket wheel 252 and the pull chord spool 232 rotate due to an unwinding of the pull chord 222. Accordingly, the manual force applied to the pull chord 222 as a result of unwinding the pull chord 222 may be transferred to the shaft 212, which in turn may rotate the penetrator assembly 300 relative to the housing to drive the penetrator assembly 300 through bone into the intraosseous space.

The pawl 256 may also be shaped and arranged within the pawl housing 268 such that when the sprocket wheel 252 rotates in a second direction (e.g., counterclockwise), which is opposite to the first direction, meshed engagement between the pawl 256 and the sprocket wheel 252 is prevented allowing the sprocket wheel 252 to rotate relative to the pawl 256, the pawl housing 268, and the shaft 212. Without meshed engagement between the pawl 256 and the sprocket wheel 252, when the sprocket wheel 252 and the pull chord spool 232 rotate in the second direction to rewind the pull chord 222 around the pull chord spool 232, the sprocket wheel 252 and the pull chord spool 232 may rotate relative to the pawl 256, the pawl housing 268 and the shaft 212. Accordingly, the penetrator assembly 300 connected to the shaft 212 may be undisturbed while the pull chord 222 is rewound about the pull chord spool 232. In alternate embodiments, the first direction may be the counterclockwise direction and the second direction may be the clockwise direction.

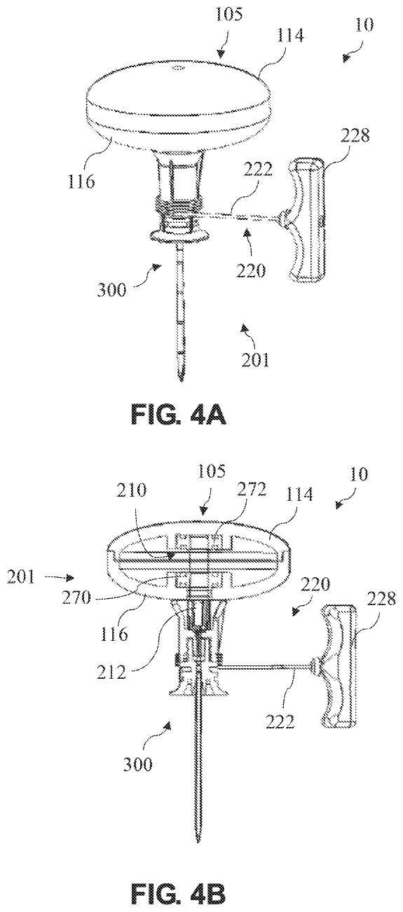

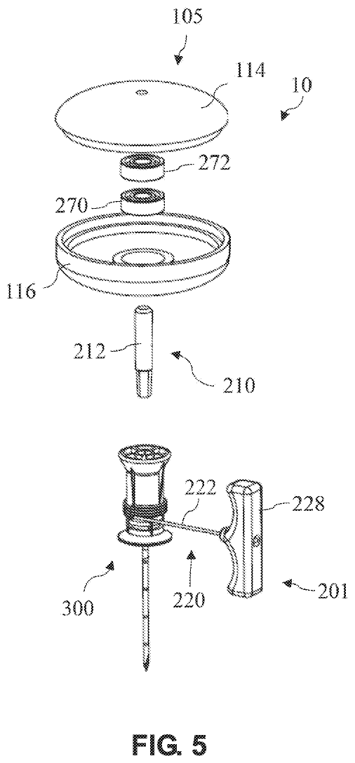

As shown in FIGS. 4A, 4B, and 5, the IO device 10 may be equipped with a single-use transmission 201. The single-use transmission 201 does not automatically rewind for multiple uses. Nevertheless, the single-use transmission 201 could be manually rewound and used again. Alternatively, the single-use transmission 201 could be disposed of after one or more uses. The pull chord 222 of the single-use transmission 201 may, for example, be wound directly around the penetrator assembly 300. The first portion 210 of the single-use transmission 201 may include a first bearing 270 and a second bearing 272, the first bearing 270 and the second bearing 272 each being disposed within the housing. The shaft 212 may extend through the first bearing 270 and the second bearing 272, and the first bearing 270 and the second bearing 272 may connect that shaft 212 to the housing in a manner that allows the shaft 212 to rotate relative to the housing. Accordingly, the manual force applied to the pull chord 222 as a result of unwinding the pull chord 222 may be transferred to directly to the penetrator assembly 300, which in turn may rotate the penetrator assembly 300 about the shaft 212 and relative to the housing to drive the penetrator assembly 300 through bone into the intraosseous space.

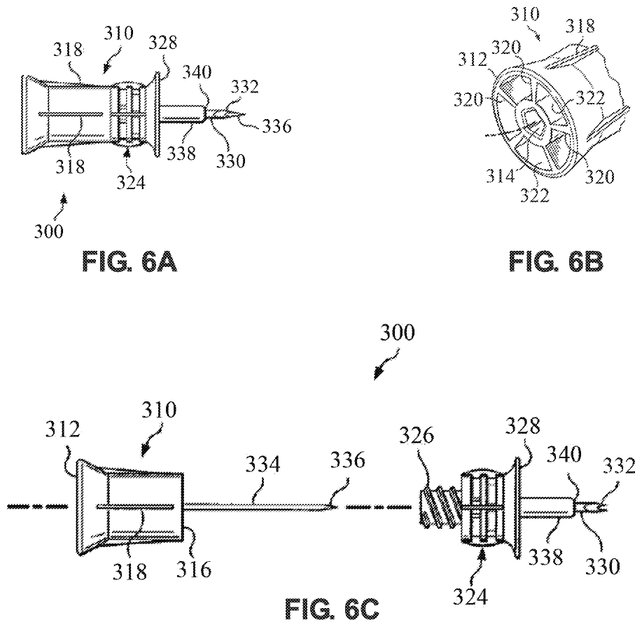

FIGS. 6A, 6B, and 6C show an exemplary embodiment of the penetrator assembly 300 of the IO device 10 according to aspects of the invention. The penetrator assembly 300 may include a connector 310, an associated hub 324, an outer penetrator 330, and an inner penetrator 334. The outer penetrator 330 may include, e.g., a cannula, hollow tube, or hollow drill bit. The inner penetrator 334 may include, e.g., a stylet or trocar. Various types of stylets and/or trocars may be disposed within the outer penetrator 330.

The connector 310 that may connect the penetrator assembly 300 to the transmission. For some applications, connector 310 may be described as having a generally cylindrical configuration defined in part by a first end 312 and a second end 316. Exterior portions of the connector 310 may include an enlarged tapered portion adjacent to first end 312. A plurality of longitudinal ridges 318 may be formed on the exterior of connector 310 to allow an operator to grasp associated penetrator assembly 300 during attachment with the shaft 212. Longitudinal ridges 318 also allow connector 310 to be grasped for disengagement from hub 324 when outer penetrator 330 has been inserted into bone and associated bone marrow.

The first end 312 of the connector 310 may include an opening 314 sized to receive portions of the shaft 212 therein. A magnet (not shown) may be disposed within the opening 314 to attract the shaft 212 to the opening 314. A plurality of webs 320 may extend radially outward from opening 314. The webs 320 cooperate with each other to form a plurality of openings 322 adjacent to first end 312. Opening 314 and openings 322 cooperate with each other to form portions of a connector receptacle.

Second end 316 of connector 310 may include an opening (not expressly shown) sized to receive first end 326 of hub 324, therein. Threads may be formed in the opening adjacent to second end 316 of connector 310. Such threads may be used to releasably attach connector 310 with threads disposed adjacent to a first end 326 of the hub 324.

The first end 326 of hub 324 may have a generally cylindrical pin type configuration compatible with releasable engagement with second end 316 of connector 310. For some applications first end 326 and threads may provide a portion of a Luer lock connection. Various types of Luer lock connections may be formed on the first end 326 of hub 324 for use in to releasably engage tubing and/or other medical devices (not expressly shown) with hub 324 after IO device 10 had been inserted into bone marrow at a target area and inner penetrator 334 removed from outer penetrator 330.

For some applications outer penetrator 330 may be described as a generally elongated tube sized to receive inner penetrator 334 therein. Portions of inner penetrator 334 may be slidably disposed within a longitudinal passageway (not expressly shown) extending through outer penetrator 330. The outside diameter of inner penetrator 334 and the inside diameter of the longitudinal passageway may be selected such that inner penetrator 334 may be slidably disposed within outer penetrator 330.

A tip 332 of the outer penetrator 330 and/or a tip 336 of inner penetrator 334 may be operable to penetrate bone and associated bone marrow. The configuration of tips 332 and/or 336 may be selected to penetrate a bone or other body cavities with minimal trauma. Tip 336 of inner penetrator 334 may be trapezoid shaped and may include one or more cutting surfaces. In one embodiment outer penetrator 330 and inner penetrator 334 may be ground together as one unit during an associated manufacturing process. Providing a matching fit allows respective tips 332 and 336 to act as a single drill, which facilitates insertion and minimizes damage as portions of penetrator assembly 300 are inserted into a bone and associated bone marrow.

Inner penetrator 334 may also include a longitudinal groove (not expressly shown) that runs along the side of inner penetrator 334 to allow bone chips and/or tissues to exit an insertion site as penetrator assembly 300 is drilled deeper into an associated bone. Outer penetrator 330 and inner penetrator 334 may be formed from stainless steel, titanium or other materials of suitable strength and durability to penetrate bone.

For some applications a depth control limiter incorporating teachings of the present disclosure, such as collar 338, may be disposed on and engaged with exterior portions of outer penetrator 330. The collar 338 may sometimes be referred to as a “depth control limiter.” Other examples of depth control limiters which may be used, or adapted for use, with the present invention may be found, e.g., in U.S. Pat. No. 8,998,848, the contents of which is hereby expressly incorporated by reference herein in its entirety.

For some embodiments collar 338 may have a generally elongated, hollow configuration compatible with engaging the outside diameter of outer penetrator 330. A first end (not expressly shown) of collar 338 may be installed over exterior portions of outer penetrator 330 and disposed within adjacent portions of hub 324. A second end 340 of collar 338 may extend a selected distance from a second end 328 of hub 324. Various techniques such as, but not limited to, press fitting may be used to install collar 338 on exterior portions of outer penetrator 330.

The resulting spacing between second end 328 of hub 324 and second end 340 of collar 338 may limit depth of penetration of outer penetrator 330 into bone and associated bone marrow. Second end 328 of hub 324 and second end 340 of collar 338 may cooperate with each other to provide a depth control limiter for associated IO device 10 or penetrator assembly 300. Collar 338 may be formed from various materials including stainless steel, titanium or other materials used to form outer penetrator 330.

Collar 338 will generally be securely engaged with the exterior of outer penetrator 330. As a result, outer penetrator 330 and collar 338 will generally rotate with each other when force is applied via the transmission. For other applications portions of the IO device 10 and an associated depth control limiter may be operable to rotate relative to each other during insertion of the IO device 10 into bone marrow adjacent to a selected insertion site.

Hub 324 may be used to stabilize the penetrator assembly 300 during insertion of an associated penetrator into a patient's skin, soft tissue and adjacent bone at a selected insertion site. Hub 324 may also be used as a handle to manipulate outer penetrator 330 or remove outer penetrator 330 from a target area. First end 326 of hub 324 may be operable for releasable engagement or attachment with associated connector 310.

Passageway (not shown) may extend from first end 326 through second end 328. The inside diameter of the passageway may be selected to securely engage the outside diameter of inner penetrator 334. The dimensions and configuration of passageway may be selected to maintain associated outer penetrator 330 engaged with hub 324.

Second end 328 of hub 324 may have a size and configuration compatible with an insertion site for an associated penetrator assembly 300.

Guide mechanisms and/or stabilizers (not shown) may be provided when the IO device 10 is inserted into a patient, to guide and/or stabilize the IO device 10 during insertion. Examples of such guide mechanisms and/or stabilizers that may be used, or adapter for use, with the present invention may be found in U.S. Pat. Nos. 9,839,740 and 8,974,410, which are each hereby expressly incorporated by references herein in their entirety.

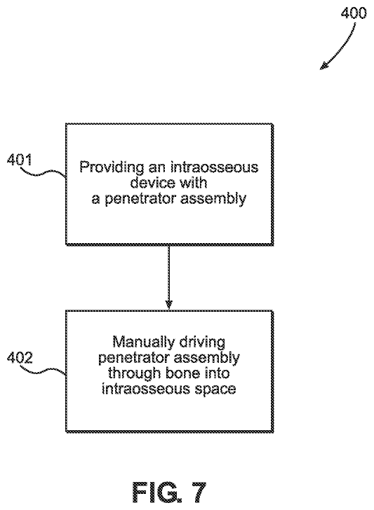

FIG. 7 illustrates an exemplary process 400 for accessing an intraosseous space of a patient in accordance with aspects of the invention. At a first step 401, the process 400 may include providing an IO device 10. The IO device 10 may include aspects of any of the above-described embodiments. For example, the IO device 10 may include the housing and the transmission at least partially disposed within the housing. The transmission may include the first portion 210 and the second portion 220. The transmission may be any of the above-described transmission embodiments including the reusable transmission 200 and/or the single-use transmission 201. The IO device 10 may further include the penetrator assembly 300 releasably connected to the first portion 210 of the transmission.

At a second step 402, the process 400 may include driving the penetrator assembly 300 through bone into the intraosseous space of the patient. Driving the penetrator assembly 300 through the bone and into the intraosseous space may include manually applying a first force to the housing. The first force may direct the IO device 10 towards an insertion site on the patient. The insertion site may be disposed on a patient at, for example, the proximal humerus, the proximal tibia, the distal tibia, etc. An operator may grasp the housing with one hand and press the housing towards the insertion site. Driving the penetrator assembly 300 through the bone and into the intraosseous space may further include manually applying a second force, concurrently with manually applying the first force, to the second portion 220 of the transmission and transmitting the second force to the penetrator assembly 300 to rotate the penetrator assembly 300 relative to the housing. For example, the operator may apply the manual force to the second portion 220 of the transmission (e.g., at the handle 228 of the pull chord 222) by unwinding the pull chord 222 of the second portion 220 of the transmission to thereby rotate the penetrator assembly 300. Upon completion of driving the penetrator assembly 300 though the bone and into the intraosseous space, the penetrator assembly 300 may be removed from the first portion 210 of the transmission permitting access to the intraosseous space to, e.g., inject fluids into the patient in a known manner.

The many features and advantages of the IO device 10 and methods described herein are apparent from the detailed specification, and thus, the claims cover all such features and advantages within the scope of this application. Further, numerous modifications and variations are possible. As such, it is not desired to limit the IO device 10 to the exact construction and operation described and illustrated and, accordingly, all suitable modifications and equivalents may fall within the scope of the claims.

What is claimed is:

1. An intraosseous device comprising:
a housing;
a transmission at least partially disposed within the housing, the transmission including a first portion and a second portion, the second portion including a manual actuator that is at least partially disposed outside of the housing; and
a penetrator assembly releasably connected to the first portion of the transmission, wherein
the transmission is configured to transmit a force, which is applied manually to the manual actuator, to the penetrator assembly to rotate the penetrator assembly relative to the housing and to drive the penetrator assembly through a bone into an intraosseous space.

2. The intraosseous device according to claim 1, wherein the manual actuator is a pull chord and the force is configured to be applied by an operator to a first end of the pull chord to unwind the pull chord and transmit the force to the penetrator assembly.

3. The intraosseous device according to claim 2, wherein the second portion of the transmission includes a handle connected to the first end of the pull chord.

4. The intraosseous device according to claim 3, wherein the transmission is a reusable transmission including a third portion that is configured to automatically retract the pull chord.

5. The intraosseous device according to claim 4, wherein
the third portion of the reusable transmission includes a pull chord spool around which the pull chord is configured to be wound, and
the pull chord spool is configured to rotate relative to the housing.

6. The intraosseous device according to claim 5, wherein
the third portion of the reusable transmission includes a spring assembly that biases the pull chord spool in a wound position at which the pull chord is wound a predetermined amount around the pull chord spool, and
the spring assembly is configured to automatically rewind the pull chord.

7. The intraosseous device according to claim 5, wherein
the first portion of the reusable transmission includes a shaft that is configured to connect to the penetrator assembly, and
the third portion of the reusable transmission includes a ratchet that connects the pull chord spool to the shaft such that the pull chord spool rotates together with and transfers the force to the shaft when the pull chord is unwound and such that the pull chord spool rotates independently from and relative to the shaft when the pull chord spool is rewound.

8. The intraosseous device according to claim 2, wherein the transmission is a single-use transmission and the pull chord is configured to be wound directly around the penetrator assembly.

9. The intraosseous device according to claim 8, wherein the first portion of the single-use transmission includes a first bearing and a second bearing each disposed within the housing.

10. The intraosseous device according to claim 9, wherein the first portion of the single-use transmission includes a shaft that extends through the first bearing and the second bearing and that is configured to connect to the penetrator assembly.

11. The intraosseous device according to claim 1, wherein the first portion of the transmission includes a shaft that is configured to connect to the penetrator assembly.

12. The intraosseous device according to claim 11, wherein the shaft is keyed.

13. The intraosseous device according to claim 12, wherein the shaft is magnetic.

14. The intraosseous device according to claim 1, wherein the housing includes an ergonomic portion.

15. The intraosseous device according to claim 14, wherein the ergonomic portion is textured.

16. The intraosseous device according to claim 14, wherein
the housing further comprising an other portion that is distinct from the ergonomic portion, the other portion comprising a first material,
the ergonomic portion comprising a second material, and
the first material is different from the second material.

17. The intraosseous device according to claim 1, wherein the housing is a ball-shaped housing.

18. The intraosseous device according to claim 17, wherein
the ball-shaped housing includes a first opening and a second opening,
the first portion of the transmission projects from the first opening and the second portion of the transmission projects from the second opening.

19. The intraosseous device according to claim 18, wherein
the first opening and the second opening are offset from each other by 90 degrees such that the first portion and the second portion of the transmission project from the ball-shaped housing perpendicularly relative to each other.

20. A method of accessing an intraosseous space of a patient comprising:
providing an intraosseous device according to claim 1;
driving the penetrator assembly through a bone into the intraosseous space of the patient comprising:
manually applying a first force to the housing, the first force directing the intraosseous device towards an insertion site on the patient; and
manually applying a second force, concurrently with manually applying the first force, to the second portion of the transmission and transmitting the second force to the penetrator assembly to rotate the penetrator assembly relative to the housing.
